(19) United States
(12) Patent Application Publication
(10) Pub. No.: US 2014/0256025 A1
(43) Pub. Date: Sep. 11, 2014

(54) METHODS AND ENZYMATIC DETERGENTS FOR REMOVING BIOFILM

(71) Applicant: RUHOF CORPORATION, Mineola, NY (US)

(72) Inventors: Marc B. Esquenet, Mineola, NY (US); Bernard E. Esquenet, Mineola, NY (US); Lee A. Ruvinsky, Mineola, NY (US); Alcides Martinez, Mineola, NY (US)

(73) Assignee: RUHOF CORPORATION, Mineola, NY (US)

(21) Appl. No.: 14/196,868

(22) Filed: Mar. 4, 2014

Related U.S. Application Data
(60) Provisional application No. 61/773,934, filed on Mar. 7, 2013.

(51) Int. Cl. C11D 3/386; A61B 19/00; C11D 3/22

(52) U.S. Cl. CPC C11D 3/38645; C11D 3/38627; C11D 3/22; C11D 3/38618; A61B 19/34; A61B 2019/343
USPC 435/264; 510/161; 134/201; 134/166 R

(57) ABSTRACT

A composition for cleaning a medical instrument having a surface at least partially covered by a biofilm. The composition may include a first enzyme having a weight of less than about 10% of the total weight of the composition, a second enzyme having a weight of less than about 10% of the total weight of the composition, and a surfactant having a weight less than about 10% of the total weight of the composition. The second enzyme may be different from the first enzyme. Additionally, the composition may have a pH ranging from about 4 to about 12, and the composition may be configured to remove greater than about 90% of the biofilm from the medical instrument in less than about 10 minutes.

### TECHNICAL FIELD

The present disclosure is directed to a detergent and, more particularly, to methods and enzymatic cleaners for removing biofilm.

### BACKGROUND

A biofilm is produced by a complex and coordinated network of microbes having increased resistance to detergents and antibiotics. Microbes within the network form an organic polymer matrix, producing a sticky mucous coating, or slime. The matrix provides structural support for cellular communities formed within the network. Channels may distribute nutrients within the network, allowing the communities to grow in a more isolated environment.

Biofilms can include a variety of microbes, including aerobic and anaerobic bacteria, algae, protozoa, and fungi. The bacteria in a biofilm can have significantly different properties from free-floating bacteria due to the complex matrix structure. For example, microbial cells within the matrix may have unique gene expression. This may allow synergistic interactions within the complex network.

Biofilms can develop and cover a wide range of surfaces including plumbing systems, sewage treatment plants, heat exchangers, dental teeth, and medical devices. The formation of biofilm on these surfaces may not only restrict fluid flow, but may also reduce the operating lifetime of the surface material. For example, biofilm growth on medical instruments is a major problem in the medical community. Biofilms may present a risk of contamination, resulting in infection and even death for patients in contact with such medical instruments. Large costs are incurred each year by health care providers to prevent and control contamination of biofilm.

Complex medical instruments, such as endoscopes, may be too costly to be disposable and are designed for re-use. Hospitals may only have a limited number of endoscopes, due to their high cost, and may be required to quickly clean and reuse an endoscope for a new patient. If the biofilm is not properly removed after each use, repeated usage can facilitate build-up of biofilm over time. Additionally, endoscopes generally include long and narrow internal channels which are exposed to tissue and fluids within patients. These long and narrow channels may be difficult to properly clean and therefore especially prone to biofilm build-up. Through and fast cleaning may be essential within a hospital environment to reduce contamination by biofilm growth.

Endoscopic reprocessors are traditionally used with a detergent to remove biofilm from an endoscope. Automated endoscopic reprocessors (AERs) are programmable devices to control and monitor cleaning parameters of an endoscope. Typically, an endoscope is placed within a basin of the AER and submerged in the detergent to clean the outer surface of the endoscope. Additionally, numerous tubes can be connected to one or more ports of the endoscope to clean the long and narrow internal channels of the endoscope. AERs also rinse and disinfect the endoscope.

Some traditional detergents, used to clean the endoscope within an AER, are designed to degrade biofilm proteins. The biofilm may then be washed away from the medical instrument. However, traditional detergents are unsatisfactory at thoroughly and quickly removing biofilm from medical instruments, such as endoscopes. Traditional detergents may leave biofilm deposits on the medical instrument or may require long contact time with the medical instrument in order to remove all the biofilm.

The present disclosure overcomes at least some of the problems associated with traditional detergents.

### SUMMARY

The present disclosure is directed to a composition for cleaning a medical instrument having a surface at least partially covered by a biofilm. The composition includes a first enzyme having a weight of less than about 10% of the total weight of the composition, a second enzyme having a weight of less than about 10% of the total weight of the composition, and a surfactant having a weight of less than about 10% of the total weight of the composition. The second enzyme may be different from the first enzyme. Additionally, the composition may have a pH ranging from about 4 to about 12, and the composition may be configured to remove greater than about 90% of the biofilm from the medical instrument in less than about 10 minutes.

The present disclosure is also directed to a method for removing at least part of a biofilm from a medical instrument. The method may include applying to the medical instrument a composition including an enzyme mixture and having a pH ranging from about 4 to about 12. Additionally, the method may include removing greater than about 90% of the biofilm from the medical instrument in less than about 10 minutes.

### BRIEF DESCRIPTION OF THE DRAWINGS

FIG. 1 is a diagrammatic representation of an automated washer according to an exemplary embodiment of the present disclosure;

FIG. 2 is a diagrammatic illustration of a circuit to produce biofilm, according to an exemplary embodiment of the present disclosure;

FIG. 3 is a graph showing the reduction of the number of viable bacteria present within the biofilm obtained using an exemplary method and detergent of the present disclosure; and

FIG. 4 is a graph showing the reduction of the residual amount of proteins present within the biofilm obtained using an exemplary method and detergent of the present disclosure.

It is to be understood that both the foregoing general description and the following detailed description are exemplary and explanatory only and are not restrictive of the disclosure, as claimed. The accompanying drawings, which are incorporated in an constitute a part of this specification, illustrate embodiments of the present disclosure and together with the description, serve to explain the principles of the disclosure.

### DETAILED DESCRIPTION

Reference will now be made in detail to the present exemplary embodiments of the present disclosure, examples of which are illustrated in the accompanying drawings. Wherever possible, the same reference numbers will be used throughout the drawings to refer to the same or like parts. Although described in relation to a detergent for cleaning a medical instrument, it is understood that the detergents and methods of the present disclosure can be employed to clean various other objects or devices. Moreover, other embodiments of the present disclosure will be apparent to those skilled in the art from consideration of the specification and practice of the present disclosure herein. It is intended that the specification and examples be considered as exemplary only, with a true scope and spirit of the present disclosure being indicated by the claims contained herein.

The present disclosure is directed to methods of at least partially removing or disrupting a biofilm present on a surface. This can include contacting the surface with an effective amount of a detergent to reduce a significant portion of the biofilm in a reduced amount of time, as discussed in more detail below. The present detergent can be used for hospital or clinical applications.

The term “surface” is defined herein as any surface which may be covered, at least in part, by a biofilm. This may include surfaces of medical devices prone to biofilm formation. Examples of surfaces may include, but are not limited to, metal, plastic, rubber, glass, or any material suitable for a medical device. In one embodiment, the surface may include an internal or external surface of a medical instrument, such as, but not limited to, an endoscope. Internal lumens of endoscopes can be prone to biofilm formation. Further, the surface may include a coating, such as, for example, polytetrafluoroethylene.

The detergent may include a solid, liquid, spray, or gel composition configured to at least partially remove biofilm from a surface. Specifically, the detergent may be configured to remove biofilm from the surface in a reduced amount of time. Unlike prior detergents, the present detergent may remove biofilm from a medical instrument in less than about 60, 30, 20, 10, or 5 minutes. The detergent may include one or more components, including an enzyme mixture and one or more surfactants. Additionally, one or more additives may be incorporated into the detergent to affect cleaning efficiency. The detergent may additionally be suitable for use with a cleaning device to remove biofilm from the medical instrument. The detergent may be directed into the cleaning device and in contact with the external and internal surfaces of the medical instrument. Additionally, the detergent may be suitable to remove biofilm from the medical instrument through manual cleaning.

The detergent may have a pH configured for biofilm removal from a medical instrument. In one embodiment, the pH may range from about 4 to about 12. Specifically, the detergent may have a pH from about 6 to about 8. In an exemplary embodiment, the detergent has a neutral pH of about 7.0.

The detergent of the present disclosure may be used at a concentration sufficient to reduce or remove biofilm from a surface. For example, the detergent may be diluted with water. In one embodiment, the detergent may be used at a concentration of from about 1 ml/L to about 16 ml/L. Specifically, the detergent may be used at a concentration of from about 2 ml/L to about 8 for commercial use. However, the detergent may be prepared in more or less concentrated forms.

The detergent may be configured to operate at temperatures ranging from about 20° C. to about 70° C. Specifically, the detergent may be used at a temperature ranging from about 25° C. to about 60° C.

The detergent may be configured to reduce a significant portion of the biofilm from a surface at least partially covered by biofilm. For example, the detergent may reduce greater than about 80%, 90%, 95%, or 99% of the biofilm from the surface in a specified time as described above.

### Enzyme Mixture

The detergent may include a plurality of enzymes. Examples of enzymes include one or more hydrolases, amylases, lipases, cellulases, carbohydrates, and any combination. Other enzymes may include proteins useful for enzymatic activity.

Hydrolases that may be used include, for example, protease, glucanases, cellulases, esterases, mannanases, and arabinases. Serine proteases, such as, for example, subtilisins may be used. Serine protease is an enzyme that catalyzes the hydrolysis of peptide bonds, and includes an essential serine residue at the active site. Other proteases that may be used include neutral proteases including, for example, aspartate and metallo-proteases. Neutral proteases have optimal proteolytic activity in a neutral pH range of about 6 to about 8, and may be derived from bacterial, fungal, yeast, plant, or animal sources.

Suitable conventional fermented commercial proteases may include, for example, Alcalase® (produced by submerged fermentation of a strain of Bacillus licheniformis), Esperase® (produced by submerged fermentation of an alkalophilic species of Bacillus), Rennilase® (produced by submerged fermentation of a non-pathogenic strain of Mucor miehei), Savinase® (produced by submerged fermentation of a genetically modified strain of Bacillus), and Durazyme® (a protein-engineered variant of Savinase®). Other commercial proteases include serine-proteases from the species Nocardiopsis, Aspergillus, Rhizopus, Bacillus alcalophilus, B. cereus, N. natto, B. vulgatus, and B. myocoide. Subtilins from Bacillus may also be used, including proteases from the species Nocardiopsis spe and Nocardiopsis dassonvillei. Metallo-proteases may include those of microbial origin, including, for example, Neutrase® (produced by submerged fermentation of a strain of Bacillus subtilis).

Amylases that may be used in the detergent include those derived from a strain of Bacillus sp. For example, the amylase may include Bacillus stearothermophilus, Bacillus amyloliquefaciens, Bacillus subtilis, or Bacillus licheniformis. Suitable Aspergillus amylases may include, for example, Aspergillus niger or Aspergillus oryzae.

Commercially suitable amylases may include, but are not limited to, those sold under the trade names Termamyl®, Stainzyme™, Duramyl™, Bioamylase D(G), Kemzym™ AT 9000, Purastar™ St, Purastar™ HPAmL, Purafect™ OxAm, Rapidase™ TEX, and Kam.

Lipases may include a microbial lipase derived from yeast, for example Candida, from bacteria, for example Pseudomonas or Bacillus, or from filamentous fungi, for example Humicola or Rhizomucor. Suitable lipases include, but are not limited to Rhizomucor miehei, Thermomyces lanuginosa, Humicola insolens, Pseudomonas stutzeri, Pseudomonas cepacia, Candida antartica, Absidia blakesleena, Absidia corymbifera, Fusarium solani, Fusarium oxysporum, Penicillum expansum, Rhodotorula glutinis, Thiarosporella phaseolina, Rhizopus microsporus, Sporobplomyces shibatanus, Aureobasidiurn puliulans, Hansenula anomala, Geotricum penicillatum, Lactobacillus curvatus, Brochothris thermosohata, Coprinus cinerius, Trichoderma harzanium, Trichoderma reesei, Rhizopus japonicas, and/or Pseudomonas plantari.

Cellulases may include any enzyme capable of degrading cellulose to glucose, cellobiose, triose, and other cellooligosaccharides. For example, the cellulose may include a endoglucanase including, but not limited to, bacterial and/or fungal endoglucanase. Examples of endoglucanases may include those obtained from the bacteria Pseudomonas or Bacillus lautus. Additionally, the cellulose may include Aspergillus niger, Aspergillus oryzae, Botrytis cinerea, Myrothecium verrucaria, Trichoderma longibrachiatum, Trichoderma reesei, Trichoderma viride, Acremonium, Aspergillus, Chaetomium, Cephalosporium, Fusarium, Gliodadium, Humicola, Irpex, Myceliophthora, Mycogone, Myrothecium, Papulospora, Penicillium, Scopulariopsis, Stachybotrys, and/or Verticillium.

Carbohydrates may include, for example, carbohydrate oxidases including glucose oxidase, hexose oxidase, xylitol oxidase, galactose oxidase, pyranose oxidase, and alcohol oxidase. Example of carbohydrate oxidases may include, but are not limited to, enzymes derived from Aspergillus niger, Cladosporium oxysporurn, Chondrus crispus, and/or Iridophycus flaccidum.

Other suitable enzymes may include xylanases, pectinases, laccases, peroxidases, phosphates, glycosidases, cellobiases, polysaccharide hydrolases, and/or oxidoreductases.

The detergent may include one or more enzymes, including, for example, at least two different enzymes. In one embodiment, the detergent may include both a hydrolases and an amylase, for example, a protease and an amylase. In another embodiment, the detergent may include a protease, an amylase, and at least one other enzyme. Suitable enzyme mixtures may include: protease, amylase, and lipase; protease, amylase, and glucanase; protease, amylase, and carbohydrate oxidase; protease, glucanase, and esterase; protease, glucanase, esterase, and mannanase.

In one embodiment, the detergent includes an enzyme mixture including a first enzyme having a weight of less than about 10% of the total weight of the detergent, and a second enzyme having a weight of less than about 10% of the total weight of the detergent. For example, in one embodiment, the first enzyme may have a weight of about 5% and the second enzyme may have a weight of about 5%. Additionally, the enzyme mixture may be present in the detergent at a concentration from about 1 weight percent (“wt %”) to about 25 wt % of the total weight of the detergent. Specifically, the enzyme mixture may be present in the detergent at a concentration from about 2 wt % to about 8 wt % of the total weight of the detergent. However, it is contemplated that the enzymes, including the enzyme mixture, may be used at higher or lower concentrations depending on the enzymatic activity of the enzymes, the exposure time of the detergent to the biofilm, and whether the detergent is in solid, liquid, spray, or gel form. Additionally, the medical instrument exposed with the detergent, and the cleaning system utilized may affect the desired concentration of the enzymes. For example, a composition may include the enzyme mixture and about 10%, 25%, 50%, 75%, or 95% water. In certain aspects, a more diluted detergent may be used with longer exposure times to reduce or removal biofilm.

The second enzyme may be different from the first enzyme. In one embodiment, the first enzyme may include a protease and the second enzyme may include an amylase, For example, the protease and amylase may each have a weight of less than about 10% of the total weight of the composition. The ratio of the wt % of the first enzyme relative to the wt % of the second enzyme may be any amount sufficient to effectively reduce or removal biofilm from a surface.

### Additional Components

The detergent may include one or more other components, including one or more surfactants. The surfactants may be present in the detergent at a concentration of less than about 10% of the total weight of the composition, for example from about 0.5 wt % to about 10 wt % of the total weight of the composition. Specifically, the surfactants may be present in the composition from about 2 wt % to about 6 wt % of the total weight of the composition. However, it is contemplated that the concentration of surfactants may vary based on the enzymatic activity of the enzymes, the exposure time of the detergent to the biofilm, whether the detergent is in solid, liquid, spray, or gel form, the medical instrument exposed with the detergent, and the cleaning system utilized.

Suitable surfactants may include either nonionic, anionic, amphoteric, cationic, or a combination of surfactants. Nonionic surfactants may include, for example, alkanolamides, amine oxides, block polymers, ethoxylated primary and secondary alcohols, ethoxylated alkyphenols, ethoxylated fatty esters, sorbitan derivatives, glycerol esters, propoxylated and ethoxylated fatty acids, alcohols, and alkyl phenols, glycol esters, polymeric polysaccharides, sulfates and sulfonates of ethoxylated alkylphenols, and polymeric surfactants. Anionic surfactants may include, for example, ethoxylated amines or amides, sulfosuccinates and derivatives, sulfates of ethoxylated alcohols, sulfates of alcohols, sulfonates and sulfonic acid derivatives, phospohate esters, and polymeric surfactants. Amphoteric surfactants may include, for example, betaine derivatives. Cationic surfactants may include, for example, amine surfactants.

Other components present in the detergent may include one or more additives. The additives may be present in the detergent from about 0.1 wt % to about 2.0 wt % of the total weight of the detergent. Suitable additives may include, but are not limited to, enhancing agents, buffers, reagents, biocides, bactericides, fungicides, bleaching agents, caustic, or biopolymer degrading agents. Additionally, the detergent may include one or more stabilizing agents, including, for example, calcium ions, magnesium ions, propylene glycol, polyethylene glycol, sodium borate, and suitable enzymes. It is further contemplated that additional additives may be used with the detergent to enhance the efficiency of the detergent.

### Cleaning Systems

The detergent of the present disclosure may be used with a variety of cleaning systems to reduce or removal biofilm from a medical instrument. For example, the detergent may be used with the cleaning systems and remove greater than about 90% of the biofilm from a medical instrument in less than about 10 minutes. In some embodiments, the detergent may be used with cleaning devices including, but not limited to, an automated washer, a washer/disinfector, a cart washer, a tunnel washer, or any other instrument washer known in the art, as will be discussed in greater detail below. Additionally, the detergent may be used with a manual cleaning system, as will also be described in greater detail below.

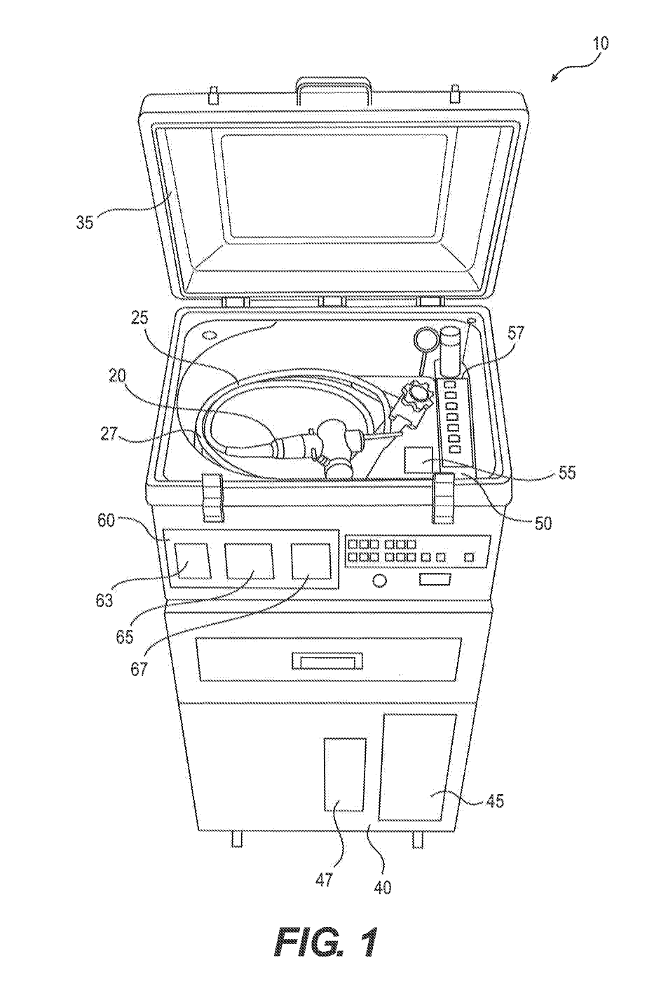

As shown in FIG. 1, an automated washer 10 may be a cleaning system, and may include one or more components to clean, rinse, or disinfect a medical instrument 20, such as an endoscope, having a biofilm. For example, the components may include a basin 30, an injection mechanism 40, a drive mechanism 50, and a controller 60. In one embodiment, automated washer 10 may include an AER device.

Medical instrument 20 may include a device configured for a use associated with a medical procedure. Medical instrument 20 may include one or more external surfaces 25 and one or more internal surfaces 27. In some embodiments, internal surfaces 27 may include one or more channels or lumens. Examples of medical instruments include, but are not limited to, endoscopes or catheters. Additionally, medical instrument 20 may include various instruments configured to deliver or extract a device from a patient, for example, a filter, surgical staple, aneurysm coil, stent, or other implantable devices.

In one example, medical instrument 20 includes an endoscope having a flexible tube and an operation portion. The flexible tube may have a length ranging from approximately 120 mm to approximately 145 mm, and a preformed bend with a radius of curvature from approximately 3 degrees to approximately 30 degrees. The flexible tube may include an inner braided material and an outer cover. The braided material may provide flexibility and the outer cover may provide protection from water. The operation portion may include a grasping portion for use by a user of medical instrument 20 and one or more operation knobs. Additionally, the endoscope may include a first lumen configured for an implantable device and a second lumen configured for imaging devices, such as, for example, fiber optic cables. The lumens may extend through the flexible tube, and may range from approximately 2 mm to approximately 6 mm in diameter.

Basin 30 may include a container configured to receive medical instrument 20. For example, medical instrument 20 may be wrapped into a coil configuration within basin 30. In the embodiment of FIG. 1, automated washer 10 includes one basin 30. However, in alternate embodiments, automated washer 10 may include multiple basins, for example, two basins. As shown in FIG. 1, basin 30 may be sealed with lid 35, and may be suitable to receive a detergent, rinse water, and a disinfectant.

Basin 30 may be fluidly connected to injection mechanism 40 to receive a detergent, disinfectant, or other solution. Injection mechanism 40 may include a first tank 45, configured to hold a detergent, and a second tank 47, configured to hold a disinfectant. Fluid may flow from first and second tanks 45, 47, into basin 30 to clean and disinfect medical instrument 20. In one embodiment, first tank 45 may hold the detergent of the present disclosure. Additionally or alternatively, injection mechanism 40 may be configured to deliver a solid, gel, spray, or other form of a detergent to basin 30.

Drive mechanism 50 may include one or pumps configured to circulate fluid within basin 30. For example, drive mechanism 50 may include circulating pump 55 configured to circulate fluid onto external surfaces 25 of medical instrument 20. Additionally, drive mechanism may include one or more supply lines 57 configured to direct fluid within internal channels 27 of medical instrument 20.

Controller 60 may include a system configured to regulate or monitor automated washer 10. For example, controller 60 may take the form of a computer system. Controller 60 may be a component of automated washer 10, or alternatively, may be part of a subsystem external to automated washer 10, Controller 60 may include one or more processors 63, one or more memories 65, and one or more input/output (I/O) devices 69. Additionally, controller 60 may include one or more additional components known in the art.

Processor 63 may include one or more processing devices configured to carry out a process for cleaning medical instrument 20. For example, processor 63 may be configured to control at least one component of automated washer 10 to perform a cleaning step, a disinfecting step, or a rinsing step. Each step may include one or more cycles extending for a predetermined duration of time and using a predetermined concentration of solution. Additionally, processor 63 may regulate or monitor the amount of detergent within basin 30 and determine if fluid should be added or removed. In some embodiments, processor 63 may be configured to regulate the temperature of a solution within basin 30 and an exposure time of the solution.

Memory 65 may include one or more storage devices configured to store instructions used by processor 63. For example, memory 65 may include one or more programs or instruction sets to permit processor 63 to control at least part of the one or more cleaning, disinfecting, or rinsing steps.

I/O device 67 may be configured to receive or transmit data. For example, I/O device 67 may include one or more digital or analog communication devices to permit controller 60 to communicate with an operator or with a machine. In one embodiment, I/O device 67 may allow controller 60 to communicate with an external computer to display the current settings.

A washer/disinfector may include any cleaning system configured to clean and disinfect a medical instrument. A cart washer may include a container configured to receive the entire medical instrument. Additionally, a tunnel washer may include a conveyor belt, wherein the medical instrument may be exposed to cleaning and drying cycles while in a container on the belt. It is further contemplated that these cleaning systems may include an injection mechanism, a drive mechanism, or a controller as described above. Manual washing may include flushing the medical instrument with detergent for a sufficient amount of time.

### Method of Use

As shown in FIG. 1, the detergent of the present disclosure may be used to clean a surface, at least partially covered by a biofilm layer, of medical instrument 20. The detergent, in one embodiment, may be stored within tank 45 of automated washer 10, and may be directed into basin 30 during a cleaning step of automated washer 10. Automated washer 10 may dilute the detergent before it is applied to medical instrument 20, or alternatively, the detergent may be diluted prior to storage within tank 45. In one example, the detergent may be diluted to about 4 ml/L. During the cleaning step, circulating pump 55 may supply the detergent to external surfaces 25 of medical instrument. Supply lines 57 may further direct the detergent within internal surfaces 27 of medical instrument 20. Controller 60 may regulate the amount of detergent within basin 30 and determine if more detergent should be added. In one example, controller 60 may determine to add an additional amount of detergent to basin 30. Automated washer 10 may remove the detergent from basin 30 through a waste line (not shown). Additionally, automated washer 10 may follow the cleaning step with a rinsing step and a disinfecting step, as is well known in the art.

Automated washer 10 may regulate the temperature of the detergent within basin 30. In one example, controller 60 may increase the temperature of the detergent of the present disclosure to, for example, approximately 47 degrees Celsius. Additionally, controller 60 may regulate the time of exposure, and may provide notice to the user when the cleaning step is finished. The detergent may be exposed to medical instrument 20 for approximately 10 minutes, in one example, within automated washer 10.

In another embodiment, the detergent of the present disclosure may be used to clean a surface of medical instrument 20 with a manual cleaning process. For example, biofilm may be removed from the surface with substantially continuous flushing of the detergent on medical instrument 20. In one embodiment, a flushing device, for example a scope valet flushing device, may provide continuous flushing on medical instrument 20 for less than about 10 minutes, and remove greater than about 90% of biofilm from the surface of medical instrument 20.

The detergent of the present disclosure may also be supplied in various types of containers for use in a hospital or other clinical setting. The container could include a screw top jar, similar to, for example, the Endozime® SLR Phase One Endoscopy Bedside Care Kit (Ruhof Corp., NY). The container could also be rectangular in form, include a snap-fit lid, or be formed of a biodegradable material. In addition to the detergent, the container could also include a sponge configured for use with manual cleaning of medical instrument 20. The sponge could include a Contoured Enzymatic Sponge (Ruhof Corp., NY), and may be pre-saturated with the detergent. Antibacterial or other types of agents may also be included in the container.

### Testing Biofilm Formation

One of the challenges of evaluating biofilm cleaning has been the general unavailability of devices and methods to reproducibly create a biofilm, and then test the ability of select detergents to remove that biofilm. As described below, a system 200 can be used to evaluate biofilm removal by controllably creating a biofilm on an inner tubular surface. This system is such as disclosed in ISO/TS 15883-5, Annex F, 2006 (Dr, Lionel Pineau), incorporated by reference in its entirety.

### Biofilm Formation

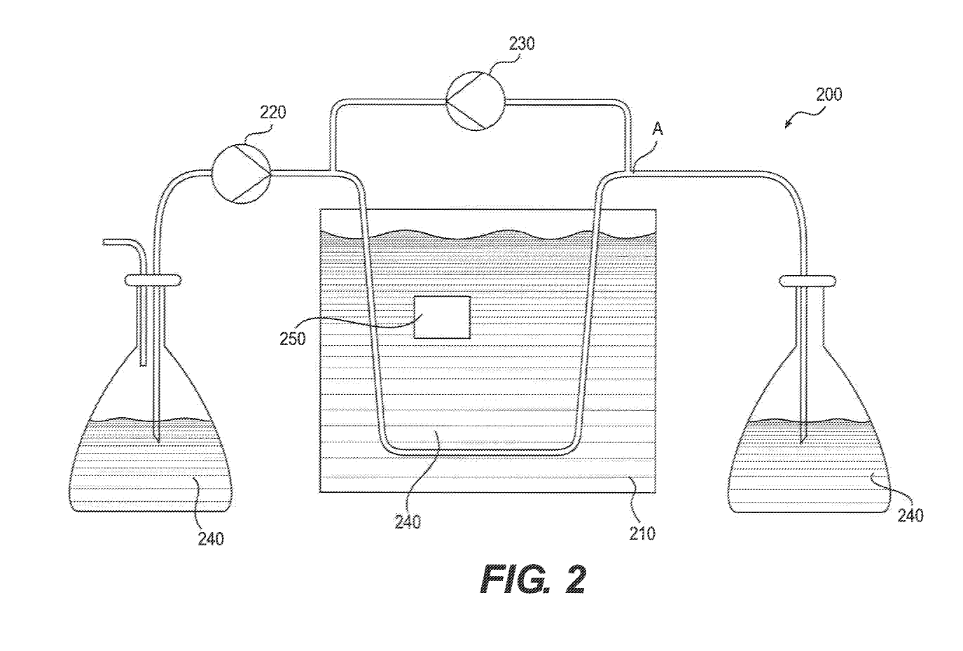

As shown in FIG. 2, system 200 may be used to prepare biofilm, and may include a water bath 210 and peristaltic pumps 220, 230. In one embodiment, pump 220 may supply system 200 with biofilm broth 240 by providing a flow of between approximately 2.5 ml/min and approximately 3.0 till/min. Pump 230 may ensure rotation of biofilm broth 240 in system 200 by providing a speed rotation of approximately 40 rpm (i.e. approximately 100 ml/min laminar flow). Test tubes 250, used for measurement of biofilm activity, may be placed in the water bath 210. Then, water bath 210 may be injected with about 5 to about 10 ml of a bacterial suspension containing approximately 10⁸ bacteria per ml, at point A. In one example, water bath 210 may be injected with the bacterial suspension while pump 220 is turned off System 200 may be maintained wider agitation for about 20 minutes, and then may be maintained in an incubator at about 30° C. for between about 72 to about 96 hours. After incubation, test tubes 250 may be removed from system 200, disinfected with alcohol, and analyzed for biofilm activity.

### Measurement of Biofilm Activity

Test tubes 250 were exposed to the detergent of the present disclosure for time segments of 5, 10, and 15 minutes. Additional test tubes 250 were exposed to sterile water to serve as controls. After each time segment, the cleaning efficacy of the detergent was evaluated by determining the number of viable bacteria still fixed on a surface of a test tube and the residual amount of proteins remaining on a surface of the test tube.

In order to determine the number of viable bacteria on a test tube, the test tube was exposed to a neutralizing agent after each time segment. This served to suspend any residual bacterial growth of the biofilm. The number of viable bacteria was then determined.

In order to determine the residual amount of proteins remaining on a test tube, the test tube was exposed to sterile distilled water after each time segment. Glass beads were then added to the water containing the test tube. This served to suspend residual proteins from the surfaces of the test tube. The residual amount of proteins was then determined using the MicroBC test method.

### Example 1

Composition A includes a 4 ml/L detergent prepared with Endozime Bio-Clean. The Control includes sterile water. Test tubes contaminated with a Pseudomonas aeruginosa biofilm were exposed to Composition A and the Control at 47° C. for 5, 10, and 15 minutes. The results are shown in Table 1.

TABLE 1

| | | Contact Time (min.) | | | |
|---|---|---|---|---|---|
| | | 0 | 5 | 10 | 15 |
| Control | Residual Viable | 8.9 | 8.6 | 8.7 | 8.7 |
| | Bacteria (Log Nb. | | | | |
| | CFU/cm²) | | | | |
| | Proteins (μg/cm²) | 57.5 | 40.2 | 39.7 | 38 |
| | Proteins (%) | 100 | 57.1 | 56.4 | 54.0 |
| Composition A | Residual Viable | 9.0 | 6.8 | 0.9 | <0.6 |
| | Bacteria (Log Nb. | | | | |
| | CFU/cm²) | | | | |
| | Proteins (μg/cm²) | 63.6 | 7.3 | 4.7 | 3.0 |
| | Proteins (%) | 100 | 11.5 | 7.3 | 4.6 |

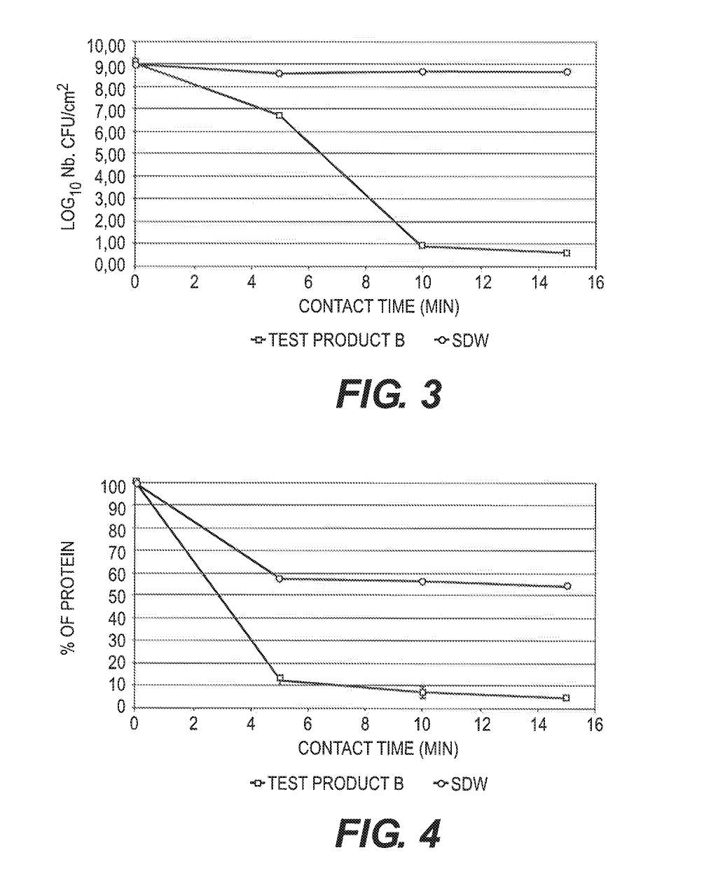

The results obtained in Table I show that Composition A induces, during the first 5 minutes, a reduction of the number of viable bacteria present within the biofilm of 2.2 log₁₀. After 10 minutes of contact with Composition A, the number of viable bacteria within the biofilm reduced by 8.1 log₁₀. Furthermore, after 15 minutes, the number of viable bacteria was lower than the limit of the detection method (i.e. <0.6 log₁₀). Comparatively, after 15 minutes of contact with the Control, the number of viable bacteria only reduced by 0.2 log₁₀. FIG. 3 shows the comparison of the number of viable bacteria, detected after contact with Composition A and with the Control.

The results obtained in Table 1 show that after contact with Composition A for 5 minutes, 88.5% of the initial amount of proteins were reduced; after contact with Composition A for 10, 92.7% of the initial amount of proteins were reduced; and after contact with Composition A for 15 minutes, 95.4% of the initial amount of proteins were reduced. Comparatively, after 15 minutes of circulation contact with the Control, the residual amount of proteins was only reduced by 46%. FIG. 4 shows a comparison of the residual amount of proteins after contact with Composition A and the Control. In summary, the data shows that the detergent of the present disclosure may effectively degrade biofilm and remove the biofilm from a surface.

As described above, the detergent of the present disclosure provides an efficient cleaner to remove biofilm from a medical instrument in reduced time. In contrast, no commercially available cleaners can remove biofilm from medical devices in such a short amount of time. Use of the present detergents and methods will allow hospitals to quickly clean endoscopes with a variety of cleaning systems. Additionally, the detergents and methods of the present disclosure may effectively remove biofilm from the medical instrument.

The present disclosure is described above with regard to an example, which is not in any way intended to limit the scope of the invention as claimed. The above example is offered to illustrate, not to limit the claimed invention.

What is claimed is:

1. A composition for cleaning a medical instrument having a surface at least partially covered by a biofilm, the composition comprising:
a first enzyme having a weight of less than about 10% of the total weight of the composition;
a second enzyme, different from the first enzyme, having a weight of less than about 10% of the total weight of the composition; and
a surfactant having a weight of less than about 10% of the total weight of the composition,
wherein the composition has a pH ranging from about 4 to about 12 and is configured to remove greater than about 90% of the biofilm from the surface in less than about 10 minutes.

2. The composition of claim 1, wherein the surfactant is selected from the group consisting of a nonionic, anionic, amphoteric, and cationic surfactant.

3. The composition of claim 1, wherein the pH is in the range from about 6 to about 8.

4. The composition of claim 1, further including at least one enzyme selected from the group consisting of lipases, cellulases, and carbohydrates.

5. The composition of claim 1, wherein the first enzyme includes a protease having a weight of less than about 5% of the total weight of the composition.

6. The composition of claim 1, wherein the second enzyme includes an amylase having a weight of less than about 5% of the total weight of the composition.

7. The composition of claim 1, wherein the composition is present in liquid form at a concentration of about 1 ml/L to about 16 ml/L.

8. A method for removing at least part of a biofilm from a medical instrument, the method comprising:
applying to the medical instrument a composition including an enzyme mixture and having a pH ranging from about 4 to about 12; and
removing greater than about 90% of the biofilm from the medical instrument in less than about 10 minutes.

9. The method of claim 8, further including applying the composition at a temperature of about 20° C. to about 70° C.

10. The method of claim 8, wherein the composition includes a concentration of approximately 4 ml/L.

11. The method of claim 8, wherein applying the composition to the medical instrument includes:
placing at least part of the medical instrument within a cleaning device; and
directing the composition into a container of the cleaning device.

12. The method of claim 11, further including storing the detergent within the cleaning device.

13. The method of claim 11, wherein the cleaning device includes a device selected from the group consisting of an automated endoscopic reprocessor, a washer/disinfector, a cart washer, and a tunnel washer.

14. The method of claim 8, further including directing the composition into at least one internal lumen of the medical instrument.

15. The method of claim 8, further including diluting the composition before applying the composition to the medical instrument.

16. The method of claim 8, further including applying the composition to the medical instrument during a cleaning step having a predetermined duration of time and a predetermined concentration of detergent.

17. The method of claim 8, wherein applying the composition to the medical instrument includes manually flushing the medical instrument with the composition.

18. A system for cleaning a medical instrument having a biofilm, comprising:
a container configured to receive at least part of the medical instrument and an enzymatic detergent; and
a processor configured to control a process for cleaning the medical instrument,
wherein the detergent is applied to the medical instrument to remove greater than about 90% of the biofilm from the medical instrument less than about 10 minutes.

19. The system of claim 18, further including a circulating pump configured to supply the detergent to an external surface of the medical instrument.

20. The device of system of 18, further including a supply line configured to direct the detergent into at least one internal channel of the medical instrument.

21. The system of claim 18, further including a tank configured to store the detergent.

22. The system of claim 18, wherein the processor is configured to regulate at least one of the amount of detergent within the basin, the temperature of a solution within the basin, and a time exposing the detergent to the medical instrument.

23. The system of claim 18, wherein the process for cleaning the medical instrument includes a cleaning step, a disinfecting step, and a rinsing step.
